SEQUENCE LISTING (7 sequences)

SEQ ID NO: 1 - DNA, 360 bp, Homo sapiens
  CDS (1)..(360): NI-301.37F1 variable heavy chain (VH) sequence
  V_region (91)..(105): complementarity determining region (CDR) VH- CDR1
  V_region (154)..(201): complementarity determining region (CDR) VH- CDR2
  V_region (298)..(327): complementarity determining region (CDR) VH- CDR3
caggtgcagctgcaggagtcgggcccaggactggtgaagccttcggagaccctgtccctcacctgcagtgtctctggtggctccatcatcagtaggagttcctactggggctggatccgccagcccccagggaaggggctggagtggattgggggtatctatcatagtgggaacacttacgacaacccgtccctcaagagtcgactcaccatgtccgtagacacgtcgaagaaccagttctccctgaatctgaggtctgtgaccgccgcagacacggctgtgtattactgtgcgaggatagtgccggggggtgatgcttttgatatctggggccaagggacaatggtcaccgtctcttcg

SEQ ID NO: 2 - protein, 120 aa, Homo sapiens
QVQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 3 - DNA, 321 bp, Homo sapiens
  CDS (1)..(321): NI-301.37F1 variable K-light chain (VK) sequence
  V_region (70)..(102): complementarity determining region (CDR) VK- CDR1
  V_region (148)..(168): complementarity determining region (CDR) VK- CDR2
  V_region (265)..(291): complementarity determining region (CDR) VK- CDR3
gacatccagatgacccagtctccatcctccctgtctgcatctgtaggagacagagtcacaatcgcttgccgggccagtcagagcgttggcacctatttaaattggtatcagcagaaaagagggaaagcccctaaactcctcatctttgctgcatccagtttgcaaagtggggtcccatcaaggttcagtggcagtggatctgggacagatttcactctcaccatcagcagtctgcaacctgaagactttgcaacttactactgtcaacagagttacagttctcctccaacgttcggccaagggaccaaggtggagatcaaa

SEQ ID NO: 4 - protein, 107 aa, Homo sapiens
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIK

SEQ ID NO: 5 - DNA, 360 bp, Homo sapiens
  CDS (1)..(360): NI-301.37F1-PIMC variable heavy chain (VH) sequence after correction of primer induced mutations
  V_region (91)..(105): complementarity determining region (CDR) VH- CDR1
  V_region (154)..(201): complementarity determining region (CDR) VH- CDR2
  V_region (298)..(327): complementarity determining region (CDR) VH- CDR3
cagctgcagctgcaggagtcgggcccaggactggtgaagccttcggagaccctgtccctcacctgcagtgtctctggtggctccatcatcagtaggagttcctactggggctggatccgccagcccccagggaaggggctggagtggattgggggtatctatcatagtgggaacacttacgacaacccgtccctcaagagtcgactcaccatgtccgtagacacgtcgaagaaccagttctccctgaatctgaggtctgtgaccgccgcagacacggctgtgtattactgtgcgaggatagtgccggggggtgatgcttttgatatctggggccaagggacaatggtcaccgtctcttcg

SEQ ID NO: 6 - protein, 120 aa, Homo sapiens
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 7 - protein, 5 aa, Artificial Sequence
  Epitope recognized by NI-301.37F1 antibody
  PEPTIDE (1)..(5): Epitope of NI-301.37F1 antibody, aa 41 to aa 45
WEPFA

(19) United States
(12) Patent Application Publication
(10) Pub. No.: US 2023/0183329 A1
(43) Pub. Date: Jun. 15, 2023

(54) COMBINATION THERAPY FOR TTR AMYLOIDOSIS

(71) Applicant: Neurimmune AG, Schlieren (CH)

(72) Inventors: Aubin MICHALON, Baden (CH); Jan GRIMM, Dübendorf (CH)

(21) Appl. No.: 17/924,496

(22) PCT Filed: May 12, 2021
(86) PCT No.: PCT/EP2021/062706

§ 371 Date: Nov. 10, 2022

(30) Foreign Application Priority Data
May 12, 2020 (EP) 20174177.4

(51) Int. Cl. C07K 16/18; A61K 31/415; A61K 31/423

(52) U.S. Cl. CPC C07K 16/18; A61K 31/415; A61K 31/423; A61K 2039/505

(57) ABSTRACT

Provided is a combination therapy for use in a method of treating transthyretin amyloidosis (ATTR) in a subject, the method comprising administering a therapeutically effect amount of an anti-transthyretin (TTR) antibody and a therapeutically effect amount of a TTR tetramer stabilizer. In addition, pharmaceutical combination products and kit of parts comprising the anti-TTR antibody and TTR tetramer stabilizer are described as well as treatment regime for their combined use in the treatment of ATTR.

This application claims priority from European Patent application EP 20174177.4 filed on May 12, 2020, the entire contents of which are incorporated herein by reference.

### FIELD OF THE INVENTION

The present invention generally relates to a combination therapy for use in a method of treating transthyretin amyloidosis (ATTR) in a subject, the method comprising administering a therapeutically effect amount of an anti-transthyretin (TTR) antibody and a therapeutically effect amount of a TTR tetramer stabilizer.

### BACKGROUND OF THE INVENTION

Transthyretin amyloidosis (ATTR) is a severe age-associated disease leading to cardiomyopathy and/or sensorimotor polyneuropathy (Gertz et al., J. Am. Coll. Cardiol. 66 (2015), 2451-24661) and includes two sub-types—wild-type ATTR (wtATTR) and variant ATTR (vATTR)—that vary regarding their pathogenesis. Their common precursor protein transthyretin (TTR) physiologically functions as a transport protein for thyroxin and retinol-binding protein. TTR is predominantly synthesized in the liver and occurs as a tetramer in its natural form (Alshehri et al., J. Neuroendocrinol. 27 (2015), 303-3239). vATTR, formerly known as hereditary/mutant ATTR, is an autosomal-dominant disorder. For both wild-type TTR (wtTTR) and mutant/variant TTR (vTTR) proteins, the pathogenic mechanism of ATTR is triggered by a partial unfolding of the TTR protein and subsequent aggregation into beta-pleated sheets forming amyloid fibrils (Eisele et al., Nat. Rev. Drug Discov. 14 (2015), 759-780).

ATTR is characterized by two main forms of clinical presentation. Predominant amyloid fibril accumulation in cardiac tissues leads to cardiomyopathy, whereas fibril deposition in nerve fibers leads to polyneuropathy (Ando et al., Guideline of transthyretin-related hereditary amyloidosis for clinicians. Orphanet J. Rare Dis. 2013; 8:31). The factors triggering amyloid deposition in a specific organ have not been elucidated yet. Patients commonly present with a mix of symptoms, with only few TTR mutations known to exclusively cause pure cardiac or neuropathic disease (Maurer et al., J. Am. Coll. Cardiol. 68 (2016), 161-172).

In the treatment of ATTR there are three current established concepts:
(i) The vTTR is almost completely replaced in the peripheral blood after orthotopic liver transplantation by the synthesis of the wtTTR in the donor liver. Of course, transplantation is not an option for a common cure of the disease.
(ii) Low molecular weight compounds stabilize the TTR tetramer and thereby minimize the formation of amyloid precursors. Diflunisal, AG10 and tafamidis stabilize the physiological TTR tetramer. Tafamidis has been approved for the treatment of stage 1 ATTR since 2011.
(iii) Gene silencers (mRNA-inhibiting oligonucleotides) reduce liver-secreted vTTR and wtTTR. Inotersen represents an antisense oligonucleotide that is administered subcutaneously (s.c.) once a week. Patisiran acts as a siRNA oligonucleotide administered intravenously (i.v.) every three weeks in combination with premedication. Both gene silencers were approved in 2018 for the treatment of ATTR stages 1 and 2.

Meanwhile, monoclonal antibodies are investigated as an additional concept of potential treatment for ATTR including anti-TTR and anti-serum amyloid P component (anti-SAP)] monoclonal antibodies.

Overviews of the different treatment strategies are given in Müller et al., European Journal of Heart Failure 22 (2020), 39-53 and Gertz et al., Brain Behav. 9 (2019), e01371.

Though each of the current drugs and concepts holds great promise to ameliorate ATTR and conditions associated therewith therapeutic strategies are still needed which address the clinical problem that early and advanced stages of ATTR may deserve different treatments and additional research is necessary to determine the optimal treatment for a given patient.

This technical problem is solved by the embodiments characterized in the claims and described further below and illustrated in the Examples and Figures.

### SUMMARY OF THE INVENTION

The present invention is based on the observation that the combined use of an anti-TTR antibody with a TTR tetramer stabilizer surprisingly shows synergistic effects in the clearance of amyloid TTR fibrils in vivo. In particular, as illustrated in the Examples, co-treatment of patient-derived amyloid TTR fibril grafts in mice with the TTR tetramer stabilizer tafamidis increases induction of the clearance of the pathological amyloid TTR fibrils by a recombinant human monoclonal anti-TTR antibody that exclusively targets the disease-associated amyloid TTR conformation with high affinity. Accordingly, the present invention generally relates to a combination therapy for use in a method of treating ATTR in a subject, the method comprising administering a therapeutically effect amount of an anti-TTR antibody and a therapeutically effect amount of a TTR tetramer stabilizer. In accordance with the present inventions, the anti-TTR antibody specifically recognizes misfolded TTR, targets with high affinity the disease-associated amyloid conformation but not physiological forms of TTR and is capable of facilitating removal of TTR amyloid, while the TTR tetramer stabilizer prevents the dissociation of TTR tetramers into amyloidogenic monomers. In accordance with the present invention, the anti-TTR antibody and the TTR tetramer stabilizer may be provided in a pharmaceutical product, for example in separate compartments and designed for the combined use as described herein below and illustrated in the appended Examples.

Co-treatment of ATTR with pharmaceuticals of different drug concepts mentioned in the background section had recently been suggested for future clinical research; see Müller et al. (2020) and Gertz et al. (2019), supra. However, as also noted by the authors though in principle many combinations may be conceivable in theory careful consideration is required, for example because of potential drug interactions.

Indeed, experiments conducted within the course of the present invention showed that binding of an anti-TTR antibody which specifically recognizes misfolded TTR, exemplified by the anti-TTR antibody NI-301.37F1, to TTR amyloid is preserved in the presence of the TTR tetramer stabilizers diflunisal, tafamidis and AG10 (see FIG. 1 and Example 1; FIG. 5A and Example 4), though the EC₅₀ value increased in the presence of diflunisal while in the presence of tafamidis the EC₅₀ value of the antibody remained substantially unaffected in kind. More importantly, thanks to applicant's novel and proprietary patient-derived amyloid xenograft (PDAX) mouse model disclosed in WO 2020/094883 A1 it could be surprisingly shown that in the presence of tafamidis the in vivo clearance of pathological TTR fibrils induced by the antibody even seemed to increase, while upon co-treatment with diflunisal pathological amyloidogenic TTR fibrils were still removed to a significant extent compared to control but the clearance rate seemed to be lower in comparison to treatment with the antibody alone, though not significantly in view of the p-value; see FIG. 2 and Example 2.

Thus, taking the additional TTR tetramer stabilizing effect into account, the experiments performed within the scope of the present invention indicate that TTR stabilizers such as tafamidis, diflunisal and AG10 are suitable for combination therapy of ATTR. However, as also shown in the Examples there may be drug interactions between anti-TTR antibody and TTR tetramer stabilizer, which may vary regarding possible supportive and synergistic effects on the clearance of pathological TTR amyloid fibrils by the anti-TTR antibody.

Without intending to be bound by theory it is believed that diflunisal because of its known anti-inflammatory properties has an immune-lowering activity which may not contribute to the anti-TTR antibody induced clearance of TTR fibrils by immune cells. Again, using applicant's proprietary PDAX mouse model disclosed in WO 2020/094883 A1 it could be validated that anti-TTR antibody mediates ATTR fibril clearance by antibody effector functions, in particular antibody-dependent cellular phagocytosis (ADCP); see Example 3 and FIG. 3 which indicate that an active Fc domain is required for antibody-mediated fibril removal by phagocytic immune cells in vivo. Accordingly, due to its immune-lowering activity diflunisal may not enhance phagocytosis promoted by the anti-TTR antibody.

Accordingly, in one embodiment of the combination therapy for ATTR of the present invention, the anti-TTR antibody has an active Fc domain and is capable of inducing ADCP and the stabilizer is substantially devoid of anti-inflammatory properties, i.e. immune-lowering activity that may not contribute to phagocytosis promoted by the anti-TTR antibody. Both activities may be tested by using the PDAX mouse model disclosed in WO 2020/094883 A1 and illustrated in the Examples.

In this context, the person skilled in the art is aware that effector function and intensity are inter alia dependent on the IgG class or isotype and that IgG2 and IgG4 have only attenuated effector functions compared to IgG1 or IgG3. Therefore, in a preferred embodiment the anti-TTR antibody used in the combination therapy of the present invention is of the IgG1 or IgG3 class or isotype, most preferably IgG1. Of course, besides using native IgG immunoglobulins corresponding effector functions can be genetically engineered; see, e.g., Saunders KO (2019) Conceptual Approaches to Modulating Antibody Effector Functions and Circulation Half-Life. Front. Immunol. 10:1296. doi: 10.3389/fimmu.2019.01296.

TTR tetramer stabilizers are small molecules that influence the rate-limiting step in the formation of amyloid fibrils, the dissociation of TTR tetramers into amyloidogenic monomers. In 1996 it was first shown that binding of a ligand to either of the binding sites for thyroxin or retinol-binding protein on TTR stabilizes its tetrameric structure and reliably prevents TTR dissociation. Tafamidis is a small molecule that belongs to the group of benzoxazole carboxylic acids, which achieve high oral bioavailability, while lacking non-steroidal anti-inflammatory drug activity. Through binding to the thyroxin binding site on TTR with high affinity and selectivity, tafamidis induces dose-dependent kinetic stabilization of wtTTR and a range of vTTR variants, e.g., V30M, V122I, etc. Diflunisal is a non-steroidal anti-inflammatory drug (NSAID) which has immune-lowering activity, and, in addition, TTR tetramer stabilizing properties.

As mentioned, the immune-lowering activity of diflunisal may not support or contribute to the anti-TTR antibody induced clearance of TTR fibrils by immune cells. Since tafamidis has not the mentioned immune-lowering activity it may be capable of supporting or enhancing anti-TTR antibody mediated removal of TTR amyloid and therefore is preferred for use in the combination therapy with an anti-TTR antibody. AG10, another TTR tetramer stabilizer, devoid of anti-inflammatory properties like tafamidis is also expected to be suitable for a combined therapy with an anti-TTR antibody against misfolded TTR and, like tafamidis to support or enhance the antibody's capability of facilitating removal of TTR amyloid fibrils.

The findings obtained in the experiments illustrated in the Examples are of high importance and of particular interest since due to the present invention, more efficient concepts for the treatment of ATTR can be provided. In particular, the ATTR treatment approaches currently known, in particular TTR tetramer stabilizers and antisense/siRNA oligonucleotides only stabilize structural and functional disease progression and are usually only effective in patients with early disease stages, but not in patients with advanced or late-onset disease.

In contrast, the combination therapy of the present invention may pave a new and additional way of treatment in that TTR tetramers are stabilized and remain in the human body in amounts sufficient to assume their physiological function and that at the same time pathological TTR amyloid is removed from the patient and its formation anew is prevented.

Further embodiments of the present invention will be apparent from the description and Examples that follow.

### BRIEF DESCRIPTION OF THE DRAWINGS

FIG. 1: Antibody NI-301.37F1 binding to amyloidogenic TTR in the presence of TTR tetramer stabilizers. Binding affinities were evaluated by ELISA in the presence of tafamidis or diflunisal at 10 μg/mL.

FIG. 2: Evaluation of combination therapies on TTR fibril elimination in vivo. A) Representative illustrations. B) Quantification of remaining TTR fibril amount, 96 hours after treatment onset, expressed as percentage of the graft area. N=6-8 mice per group, with 4 non-consecutive sections per mouse. ** p<0.01, **** p<0.001.

FIG. 3: Antibody-mediated fibril removal activity in vivo requires an active Fc domain. ATTR fibril-grafted mice received a single administration of ch.NI-301.37F1, ch.NI-301.37F1-LALAPG or isotype control at 5 mg/kg iv, and skin biopsies were collected 5 days later for histological analysis. A) Representative illustrations. B) Quantification of remaining TTR fibril amount, 5 days after fibril graft and treatment administration, ATTR fibrils covered 46% of the graft area in isotype treated mice, 23.6% in ch.NI-301.37F1 treated mice, and 46.5% in mice treated with ch.NI-301.37F1-LALAPG. In contrast, no fibril removal activity was observed in mice treated with ch.NI-301.37F1-LALAPG compared to isotype treated mice.

FIG. 4: NI-301.37F1 binding selectivity in presence of tafamidis and diflunisal using BLI. Representative illustration of NI-301.37F1 binding to A) soluble ATTR oligomers (mis.WT-TTR) and the absence of NI-301.37F1 binding to B) TTR tetramers (tetr.WT-TTR).

FIG. 5: NI-301.37F1 binding selectivity in presence of tafamidis and AG10 using BLI. Representative illustration of NI-301.37F1 binding to A) soluble ATTR oligomers (mis.WT-TTR) and the absence of NI-301.37F1 binding to B) TTR tetramers (tetr.WT-TTR).

### DETAILED DESCRIPTION OF THE INVENTION

The present invention generally relates to a combination therapy for use in a method of treating transthyretin amyloidosis (ATTR) in a subject, the method comprising administering a therapeutically effect amount of an anti-TTR antibody and a therapeutically effect amount of a TTR tetramer stabilizer. More specifically, the present invention relates to the embodiments as characterized in the claims, disclosed in the description and illustrated in the Examples and Figures further below.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-0-19852917-0.

When using the term “ATTR”, usually vATTR as well as wtATTR is meant if not indicated otherwise. Similarly, if not indicated otherwise, “TTR” also refers to wtTTR and vTTR. The term “anti-TTR antibody” usually refers to an antibody that binds misfolded/aggregated TTR and not the physiological TTR tetramer.

The terms “combination therapy” or “combined treatment” or “in combination” or “combined pharmaceutical product” as used herein denotes any form of concurrent or parallel treatment with at least two distinct therapeutic agents, i.e. the anti-TTR antibody and TTR tetramer stabilizer.

In the context of the present application, “co-administration” of or “co-treatment” with two or more compounds is defined as administration of the two or more compounds to the patient within a specific time, usually about 24 h, including separate administration of two medicaments each containing one of the compounds as well as simultaneous administration whether or not the two compounds are combined in one formulation or whether they are in two separate formulations.

By “therapeutically effective amount” or “clinically active concentration” of a substance, it is meant that a given substance is administered to a subject suffering from a condition, in an amount sufficient to ensure, alleviate or partially arrest the condition or one or more of its symptoms. Such therapeutic treatment may result in a decrease in severity of disease symptoms, or an increase in frequency or duration of symptom-free periods. Effective amounts for a given purpose and a given agent will depend on the severity of the disease or injury as well as the weight and general state of the subject. As used herein, the term “subject” includes any mammal, preferably a human. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀.

Furthermore, unless stated otherwise, terms and expressions used herein in order to characterize the present invention, in particular the anti-TTR antibody as used in the combination product of the present invention are given in the definitions as provided in WO 2015/092077 A1, in particular in subsection “I. Definitions” at pages 16 to 42, the disclosure content of which is explicitly incorporated herein by reference. The same applies to the general embodiments disclosed in WO 2015/092077 A1 for antibodies, pharmaceutical compositions, etc.

The pharmaceutical product comprises at least two different components, an anti-TTR antibody which specifically targets misfolded human TTR and removes amyloidogenic TTR fibrils typically due to an active Fc domain which is capable of inducing antibody-dependent cellular phagocytosis (ADCP) and a TTR tetramer stabilizer such as those described in Müller et al. (2020) and Gertz et al. (2019), supra, which preferably is devoid of anti-inflammatory properties. As discussed above, the antibody is preferably of the IgG1 class or isotype.

As described above, first experiments revealed that TTR tetramer stabilizers do not interfere with binding of an anti-TTR antibody to amyloidogenic TTR. Accordingly, it is prudent to expect that any anti-TTR antibody which is capable of specifically binding to misfolded TTR, does not target physiological forms of TTR and has an active Fc domain, is suitable as component for the combination therapy of the present invention.

Since the pharmaceutical product of the present invention is used in the treatment of ATTR in human subjects, in one embodiment the anti-TTR antibody is humanized, preferably human-derived and non-immunogenic in human.

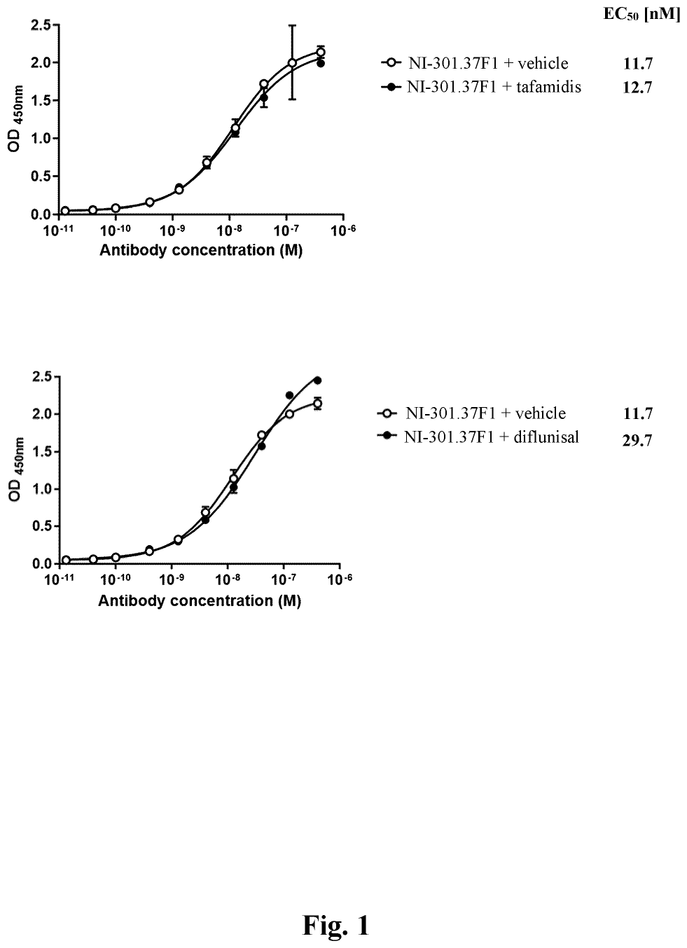

As illustrated in the Examples, the present invention has been exemplified with anti-TTR antibody NI-301.37F1 which is capable of binding a human TTR epitope which comprises or consists of the amino acid sequence TTR₄₁₋₄₅ (SEQ ID NO: 51 of WO 2015/092077 A1) and comprises in its variable region or binding domain the complementary determining regions (CDRs) and variable heavy (V_{H}) and variable light (V_{L}) chain having the amino acid sequences depicted in FIGS. 1C and 1M, respectively, of WO 2015/092077 A1. This antibody and cognate antibodies have been derived from memory B cell repertoire of healthy aged human donors; see description of the Examples in WO 2015/092077 A1. Characterization of the binding properties of the antibody demonstrated that it presents high binding affinity to misfolded TTR in the sub-nanomolar range, is highly selective for the amyloid conformation of TTR, and exerts similar binding to wtTTR and vTTR. Since this human-derived antibody is selective for misfolded TTR and as illustrated in the Examples can trigger cardiac amyloid removal it represents the most favorable therapeutic candidate for the combination therapy of the present invention.

However, WO 2015/092077 A1 discloses further human-derived antibodies that could be shown to bind to the same human TTR epitope as NI-301.37F1, i.e. antibody NI-301.28B3 and NI-301.12D3 which V_{H} and V_{L} chain amino acid sequences including indication of the CDRs are depicted for NI-301.28B3 in FIG. 1E and for NI-301.12D3 in FIG. 1L of WO 2015/092077 A1. Accordingly, the anti-TTR antibody for use in the combination therapy of the present invention may be generally characterized by binding a human TTR epitope which comprises or consists of the amino acid sequence TTR₄₁₋₄₅ (SEQ ID NO: 51 of WO 2015/092077 A1). Hence, in order to arrive at such antibodies there is no need to take recourse of the means and methods disclosed in WO 2015/092077 A1 for obtaining such antibodies anew but it is possible to perform one or more amino acid substitutions which leave the binding characteristics of the antibody unaffected in kind. In addition, any of the other human-derived antibodies disclosed in WO 2015/092077 A1 may be used as well.

Another class of anti-TTR antibodies presumably suitable for the combination therapy of the present invention is described in international applications by Prothena Biosciences Limited (Prothena). In particular, Prothena reports on an investigational monoclonal antibody designed to specifically target and clear the misfolded (toxic) forms of the TTR amyloid protein found in ATTR, wherein said antibody is designated PRX004 and is currently in a Phase 1 study in patients with ATTR (ClinicalTrials.gov Identifier: NCT03336580). According to a presentation by Jeffrey Zonder, Karmanos Cancer Institute, Detroit, in 2018 antibody PRX004 corresponds to and is the humanized version of antibody 14G8 described in Higaki et al., Amyloid 23 (2016) 86-97 and which is disclosed in WO 2016/120810 A1 and WO 2018/007922A2 and more specifically in WO 2019/108689 A1. Accordingly, antibody PRX004 would be another preferred anti-TTR antibody for use in the combination therapy of the present invention among others which recognize the same epitope as PRX004, i.e. amino acids TTR₈₉₋₉₇ or an epitope comprising amino acids TTR₁₀₁₋₁₀₉, and which are humanized versions of the originally cloned mouse monoclonal antibodies 14G8, 9D5, 5A1, 6C1 disclosed in WO 2016/120810 A1, WO 2018/007924 A2, WO 2018/007924 A2 and WO 2018/007923 A1.

A further suitable antibody is a humanized version of antibody 18C5 or an isolated monoclonal antibody that competes for binding to human TTR with monoclonal antibody 18C5, preferably that binds to the same epitope on human TTR as a monoclonal antibody 18C5, wherein 18C5 is a mouse antibody characterized by a mature heavy chain variable region having an amino acid sequence comprising SEQ ID NO: 81 and a mature light chain variable region having an amino acid sequence comprising SEQ ID NO: 87 as disclosed in WO 2019/071205 A1.

Still another class of humanized anti-TTR antibodies presumably suitable for the combination therapy of the present invention is described in international applications by The Chemo-Sero-Therapeutic Research Institute and KM Biologics Co., Ltd., respectively, recognizing an epitope comprising amino acids TTR₇₈₋₈₉ or TR₁₁₈₋₁₂₂ as disclosed for antibodies 371M and 313M in WO 2015/115332 and for the antibody described in WO 2015/115331 A1 (which is designated herein as XY for ease of reference).

Since there are two forms of ATTR, namely wtATTR and vATTR which are caused by pathological aggregation of wtTTR and vTTR, respectively, in a preferred embodiment, the antibody binds amyloidogenic wtTTR as well as amyloidogenic vTTR. More than 120 mutations in the TTR gene affect persons of all ages with vATTR, but the most common ones are the Val30Met and the Val122Ile substitution.

The Val30Met substitution is endemic in certain regions of Portugal, Sweden, and Japan and is the most frequent mutation causing ATTR polyneuropathy, previously named Familial Amyloid Polyneuropathy. Furthermore, TTR toxicity is observed as a consequence of the Val122Ile mutation, which is found with high frequency (3-5%) in the African-American and West African populations. This mutation is associated with ATTR cardiomyopathy, previously named Familial Amyloid Cardiomyopathy, a condition where massive TTR accumulation in the myocardium leads to cardiac weaknesses and ultimately cardiac failure (Ruberg et al., Circulation. 126 (2012), 1286-1300).

Accordingly, in a preferred embodiment, the anti-TTR antibody binds to a TTR epitope which does not cover amino acids Val122 and/or Val30 or the anti-TTR antibody binds to an epitope covering amino acids Val122 and/or Val30, but the amino acid substitution to Met and Ile, respectively does not affect binding of the antibody. Alternatively, the antibody binds to vTTR having different mutations, but in particular to vTTR having the Val122Ile and/or the Val30Met mutation.

Furthermore, in 10-15% of individuals over the age of 65 cardiac TTR deposits due to wtATTR are found. Thus, in a further embodiment, the anti-TTR antibody binds to aggregated wtTTR. Preferably, the anti-TTR antibody binds to wtTTR and vTTR, preferably to the same extent.

As mentioned above, the experiments illustrated in the Examples have been performed with antibody NI-301.37F1 which is disclosed in WO 2015/092077 A1 and binds to a TTR epitope comprising the amino acid sequence TTR₄₁₋₄₅ as set forth in SEQ ID NO: 51 of WO 2015/092077 A1. Accordingly, it is prudent to expect that further antibodies binding an epitope comprising said amino acid sequence are also a suitable component of the pharmaceutical product of the present invention, e.g., antibodies NI-301.28B3 and NI-301.12D3 as also disclosed in WO 2015/092077 A1.

Thus, in a particular preferred embodiment of the present invention the anti-TTR antibody is derived from human antibody NI-301.37F1, NI-301.28B3 or NI-301.12D3 and characterized by comprising in its variable region, i.e. binding domain the complementarity determining regions (CDRs) of the variable heavy (V_{H}) and variable light (V_{L}) chain having the amino acid sequences depicted in FIG. 1C [NI-301.37F1], 1E [NI-301.28B3] or 1L [NI-301.12D3] of WO 2015/092077 A1, or wherein one or more of the CDRs may differ in their amino acid sequence from those set forth in FIG. 1C, 1E or 1L of WO 2015/092077 A1 by one, two, three or even more amino acids in case of CDR2 and CDR3, and wherein the antibody displays substantially the same or identical characteristics of anti-TTR antibody NI-301.37F1, NI-301.28B3 or NI-301.12D3 illustrated in the Examples of WO 2015/092077 A1. The positions of the CDRs are shown in FIG. 1C, 1E or 1L and explained in the Figure legend to FIG. 1 in WO 2015/092077 A1. The corresponding nucleotide sequences are set forth in Table II at page 70 of WO 2015/092077 A1 for NI-301.37F1, at pages 70 to 71 for NI-301.28B3 and at page 73 for NI-301.12D3. In addition or alternatively, the framework regions or complete V_{H} and/or V_{L} chain are 80% identical to the framework regions depicted in FIG. 1C or 1M [NI-301.37F1], 1E [NI-301.28B3] or 1L [NI-301.12D3] of WO 2015/092077 A1, preferably 85%, 90%, 95%, 96%, 97%, 98%, 99% or 100% identical to the framework regions and V_{H} and/or V_{L} chain, respectively, depicted in FIG. 1C or 1M, 1E or 1L of WO 2015/092077 A1. Furthermore, cloning and expression of antibodies NI-301.37F1, NI-301.28B3 and NI-301.12D3 has been performed as described in WO 2015/092077 A1 in Examples 1 and 2 at pages 110 to 112 which methods are incorporated herein by reference.

Thus, in one embodiment the anti-TTR antibody is characterized by the CDRs of the V_{H} and V_{L} chain and by the entire VH and VL chain, respectively depicted in FIGS. 1C and 1M of WO 2015/092077 A1. Thus, the antibody preferably comprises
(i) a variable heavy (VH) chain comprising the following VH complementary determining regions (CDRs) 1, 2, and 3, and/or a variable light (VL) chain comprising the following VL CDRs 1, 2, and 3:
   (a) VH-CDR1: positions 31-35 of SEQ ID NO: 10 of WO 2015/092077 A1 or a variant thereof, wherein the variant comprises one or two amino acid substitutions,
   (b) VH-CDR2: positions 52-67 of SEQ ID NO: 10 of WO 2015/092077 A1 or a variant thereof, wherein the variant comprises one or two amino acid substitutions,
   (c) VH-CDR3: positions 100-109 of SEQ ID NO: 10 of WO 2015/092077 A1 or a variant thereof, wherein the variant comprises one or two amino acid substitutions,
   (d) VL-CDR1: positions 24-34 of SEQ ID NO: 12 of WO 2015/092077 A1 or a variant thereof, wherein the variant comprises one or two amino acid substitutions,
   (e) VL-CDR2: positions 50-56 of SEQ ID NO: 12 of WO 2015/092077 A1 or a variant thereof, wherein the variant comprises one or two amino acid substitutions, and
   (f) VL-CDR3: positions 89-97 of SEQ ID NO: 12 of WO 2015/092077 A1 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 10 or 53, or a variant thereof, wherein the variant comprises one or more amino acid substitutions; and
   (b) the VL chain comprises the amino acid sequence depicted in SEQ ID NO: 12, or a variant thereof, wherein the variant comprises one or more amino acid substitutions;
      preferably wherein the VH and VL chain amino acid sequence is at least 90% identical to SEQ ID NO: 10 or 53 and 12, respectively.

This preferred embodiment, i.e. the use of antibody NI-301.37F1 and equivalents thereof illustrated in the Examples corresponds to the embodiment characterized in claim 4, wherein reference is made to the amino acid sequences depicted in the present sequence listing.

In one embodiment the anti-TTR antibody is characterized by the CDRs of the V_{H} and/or V_{L} chain and by the entire VH and VL chain, respectively depicted in FIG. 1E of WO 2015/092077 A1. Thus, the antibody preferably comprises
(i) a variable heavy (VH) chain comprising the following VH complementary determining regions (CDRs) 1, 2, and 3, and/or a variable light (VL) chain comprising the following VL CDRs 1, 2, and 3:
   (a) VH-CDR1: positions 31-37 of SEQ ID NO: 18 of WO 2015/092077 A1 or a variant thereof, wherein the variant comprises one or two amino acid substitutions,
   (b) VH-CDR2: positions 52-67 of SEQ ID NO: 18 of WO 2015/092077 A1 or a variant thereof, wherein the variant comprises one or two amino acid substitutions,
   (c) VH-CDR3: positions 100-116 of SEQ ID NO: 18 of WO 2015/092077 A1 or a variant thereof, wherein the variant comprises one or two amino acid substitutions,
   (d) VL-CDR1: positions 24-34 of SEQ ID NO: 20 of WO 2015/092077 A1 or a variant thereof, wherein the variant comprises one or two amino acid substitutions,
   (e) VL-CDR2: positions 50-56 of SEQ ID NO: 20 of WO 2015/092077 A1 or a variant thereof, wherein the variant comprises one or two amino acid substitutions, and
   (f) VL-CDR3: positions 89-98 of SEQ ID NO: 20 of WO 2015/092077 A1 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 18, or a variant thereof, wherein the variant comprises one or more amino acid substitutions; and
   (b) the VL chain comprises the amino acid sequence depicted in SEQ ID NO: 20, or a variant thereof, wherein the variant comprises one or more amino acid substitutions;
      preferably wherein the VH and VL chain amino acid sequence is at least 90% identical to SEQ ID NO: 18 and 20, respectively.

In one embodiment the anti-TTR antibody is characterized by the CDRs of the V_{H} and/or V_{L} chain and by the entire VH and VL chain, respectively depicted in FIG. 1E of WO 2015/092077 A1. Thus, the antibody preferably comprises
(i) a variable heavy (VH) chain comprising the following VH complementary determining regions (CDRs) 1, 2, and 3, and/or a variable light (VL) chain comprising the following VL CDRs 1, 2, and 3:
   (a) VH-CDR1: positions 31-35 of SEQ ID NO: 46 of WO 2015/092077 A1 or a variant thereof, wherein the variant comprises one or two amino acid substitutions,
   (b) VH-CDR2: positions 50-66 of SEQ ID NO: 46 of WO 2015/092077 A1 or a variant thereof, wherein the variant comprises one or two amino acid substitutions,
   (c) VH-CDR3: positions 99-108 of SEQ ID NO: 46 of WO 2015/092077 A1 or a variant thereof, wherein the variant comprises one or two amino acid substitutions,
   (d) VL-CDR1: positions 23-36 of SEQ ID NO: 48 of WO 2015/092077 A1 or a variant thereof, wherein the variant comprises one or two amino acid substitutions,
   (e) VL-CDR2: positions 52-58 of SEQ ID NO: 48 of WO 2015/092077 A1 or a variant thereof, wherein the variant comprises one or two amino acid substitutions, and
   (f) VL-CDR3: positions 91-100 of SEQ ID NO: 48 of WO 2015/092077 A1 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 46, or a variant thereof, wherein the variant comprises one or more amino acid substitutions; and
   (b) the VL chain comprises the amino acid sequence depicted in SEQ ID NO: 48, or a variant thereof, wherein the variant comprises one or more amino acid substitutions;
      preferably wherein the VH and VL chain amino acid sequence is at least 90% identical to SEQ ID NO: 46 and 48, respectively.

However, preferably antibody NI-301.37F1 is used as one component in the combination therapy of the present invention.

As mentioned before, the anti-TTR antibody comprises a human constant domain with an active Fc domain and has an IgG format, i.e. being a full IgG antibody, preferably an IgG1 antibody or isotype. Recombinant expression of complete human IgG1 antibodies with a human constant domain can be performed substantially as described in, e.g., in Harlow and Lane “Antibodies, A Laboratory Manual”, CSH Press, Cold Spring Harbor (1988) First edition; Second edition by Edward A. Greenfield, Dana-Farber Cancer Institute © 2014, ISBN 978-1-936113-81-1 and the Examples of WO 2012/080518 A1.

As mentioned above, ATTR can be either wtATTR or vATTR and thus, a treatment approach for both variants is preferred. Since as described above, the anti-TTR antibody binds both aggregated wtTTR and vTTR, the second component in the combination therapy of the present inventions preferably stabilizes both TTR forms to physiologically functional tetramers.

Most pharmaceuticals currently available on the market for the treatment of ATTR have been approved for vATTR-polyneuropathy, for example inotersen and patisiran, but only tafamidis and diflunisal have been shown to be efficient for the treatment of vATTR and wtATTR; see Müller et al. (2020), supra, wherein tafamidis has been approved by the European Medicines Agency (EMA) and the U.S. Food and Drug Administration (FDA) for the treatment of ATTR-polyneuropathy and ATTR-cardiomyopathy. Furthermore, AG10 is a novel, potent, and selective oral TTR tetramer stabilizer being developed to treat wtATTR and vATTR; see Fox et al., Clinical Pharmacology in Drug Development 9 (2020), 115-129. Thus, only TTR tetramer stabilizers are clinically approved for the treatment of vATTR and wtATTR and have high potency for the respective approval in case of AG10.

Accordingly, in one embodiment, the combination therapy of the present invention further comprises one or two TTR tetramer stabilizers, preferably one TTR tetramer stabilizer which is able to stabilize wtTTR tetramers and vTTR tetramers.

As mentioned above, TTR tetramer stabilizers block tetramer dissociation, the rate-limiting step in the amyloidogenic process and reduce de novo deposition of amyloid. In 1996 it was first shown that binding of a ligand to either of the binding sites for thyroxin or retinol-binding protein on TTR stabilizes its tetrameric structure and reliably prevents TTR dissociation. Thus, the combination therapy of the present invention comprises a TTR tetramer stabilizer that binds to either of the binding sites for thyroxin or retinol-binding protein of the TTR tetramer.

In a preferred embodiment, the TTR tetramer stabilizer is tafamidis (Cas Registry number: 594839-88-0). Tafamidis is a member of the class of 1,3-benzoxazoles that is 1,3-benzoxazole-6-carboxylic acid in which the hydrogen at position 2 is replaced by a 3,5-dichlorophenyl group. Tafamidis is structurally similar to diflunisal, but in contrast to diflunisal is not an NSAID.

In a further embodiment, the combination therapy of the present invention comprises next to the anti-TTR antibody the TTR tetramer stabilizer AG10. AG10 is a 3-(3-(3,5-Dimethyl-1H-pyrazol-4-yl)propoxy)-4-fluorobenzoic acid (Cas Registry Number: 1446711-81-4) and is disclosed for example in U.S. Pat. No. 9,169,214 B2, where it is designated as compound VIIc. AG10 is a potent, highly selective, small-molecule TTR tetramer stabilizer. It is manufactured by a simple synthetic route, and its pharmaceutical properties include good oral bioavailability, high binding selectivity, and ability to stabilize TTR. AG10 was designed to mimic the structural influence of the protective TTR mutation T119M. Compared with other known stabilizers, AG10 is unique in its capacity to form hydrogen bonds with the same serine residues at position 117 that stabilize the T119M variant. First clinical trials have already been conducted and it was shown that AG10 has the potential to be a safe and effective treatment for patients with either vATTR or wtATTR (Fox et al., Clinical Pharmacology in Drug Development 9 (2020), 115-129). In particular, phase II clinical trials showed its potency for the treatment of patients with ATTR-cardiomyopathy (Judge et al., J Am Coll Cardiol 74 (2019), 285-295). However, it is prudent to expect that it will work for patients having ATTR-polyneuropathy as well.

In a further embodiment, the combination therapy of the present invention comprises next to the anti-TTR antibody the TTR tetramer stabilizer diflunisal. Diflunisal is a non-steroidal anti-inflammatory drug (NSAID) and is described in Müller et al., European Journal of Heart Failure 22 (2020), pages 39-53.

The anti-TTR antibody as part of the combination therapy of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected by different ways, e.g., by intravenous, intraperitoneal, subcutaneous, intramuscular, intranasal, topical or intradermal administration or spinal or brain delivery. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier. In a preferred embodiment, the anti-TTR antibody is formulated in a liquid formulation and is designed to be administered intravenously (i.v.) or subcutaneously (s.c).

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. Progress can be monitored by periodic assessment.

Whether or not the anti-TTR antibody for use in accordance with the present invention may be therapeutically effective can be determined by using the PDAX animal model described in Example 4 of WO 2020/094883 A1. Preferably, the anti-TTR antibody is effective when administered at a dose of at least 0.5 mg/kg, preferably at 5 or 50 mg/kg or any dose between. More preferably, the antibody is administered at a dose of about 3, 10, 30 or 60 mg/kg or any dose between every two to four weeks.

As regards the TTR tetramer stabilizers, preferably tafamidis, diflunisal and/or AG10 are used in the combination therapy of the present invention. Currently, two preparations, namely tafamidis meglumine and tafamidis are available, both which have the same active moiety, tafamidis. The TTR tetramer stabilizers are preferably administered in a clinically active concentration.

Tafamidis meglumine, administered as an 80 mg, once-daily dose (4×20 mg capsules), is approved in the United States, Japan, Canada, and Brazil for the treatment of wtATTR- and vATTR-cardiomyopathy in adults. An alternative single solid oral dosage formulation (tafamidis 61 mg free acid capsules) was developed and introduced for patient convenience (approved in the United States, United Arab Emirates, and European Union); see Lockwood et al., Clin. Pharmacol. Drug Dev., 2020 Mar. 20. doi: 10.1002/cpdd.789 as well as product information provided by the FDA as regards VYNDAQEL® (tafamidis meglumine) and VYNDAMAX™ (tafamidis). Furthermore, tafamidis meglumine, administered as an 20 mg, once-daily dose (corresponds to 12.2 mg tafamidis), is approved in more than 40 countries worldwide for the treatment of adults with early-stage symptomatic wtATTR- and vATTR-polyneuropathy; see Lockwood et al. 2002 and the product information of the EMA as regards VYNDAQEL®.

Thus, in one embodiment, tafamidis is designed to be administered at a once-daily dose of 20 mg as tafamidis meglumine or at a once-daily dose of 12.2 mg as tafamidis, preferably wherein this dosage is administered to patients having symptomatic wtATTR- and vATTR-polyneuropathy.

In one embodiment, tafamidis is designed to be administered at a once-daily dose of 80 mg as tafamidis meglumine or at a once-daily dose of 61 mg as tafamidis, preferably wherein this dosage is administered to patients having symptomatic wtATTR- and vATTR-cardiomyopathy.

In a further embodiment of the present invention, tafamidis or tafamidis meglumine are designed to be administered at a dose of 1, 5, 15, or 30 mg/kg/day or any dose in between.

In a further embodiment of the present invention, diflunisal is administered at a dose up to 250 mg once or twice daily.

AG10 is not yet approved by the regulatory agencies but first clinical trials suggest that AG10 is already effective when administered at a single dose of 50, 150, 300, 400 or 800 mg. Preferably, one single dose of 300 mg or 800 mg is used; see Fox et al. 2020 and Judge et al. 2019). In one embodiment of the present invention, AG10 is administered at a dose of 50, 150, 300, 400 or 800 mg, preferably of 400 mg or 800 mg, preferably twice daily. Of course, also doses between these amounts are encompassed by the present invention.

In one embodiment of the combination therapy of the present invention, the anti-TTR antibody and the TTR tetramer stabilizer are provided in a pharmaceutical product, wherein they are typically present in separate containers. As mentioned above, the anti-TTR antibody is preferably a liquid formulation designed to be administered intravenously. Thus, in case of the anti-TTR antibody, the container is preferably a prefilled syringe, injection pen, ampoule, bottle, autoinjector or an infusion bag. In a further preferred embodiment, the container is an infusion bottle or bag.

In contrast, the TTR tetramer stabilizer is preferably formulated for oral administration and preferably present in a tablet or capsule as described above.

Hence, in a further aspect the present invention relates to pharmaceutical product which comprises the anti-TTR antibody and the TTR tetramer stabilizer as defined hereinabove with the mentioned preferences, in particular wherein the antibody is capable of facilitating removal of amyloidogenic TTR due to an active Fc domain and is capable of inducing antibody-dependent cellular phagocytosis (ADCP) and the stabilizer is substantially devoid of anti-inflammatory properties.

In one embodiment of the present invention, the separate components of the pharmaceutical product of the present invention, i.e. at least the anti-TTR antibody and the TTR tetramer stabilizer are present in one composition, wherein the formulation and the dose regime have to be adapted in that both components remain stable and are pharmaceutically active. This embodiment may be useful clinical situations like an instant heart or stroke, where, as a first aid simultaneous administration of the two drugs could be considered.

The present invention also provides a pharmaceutical pack or kit comprising one or more containers filled with at least two of the above described ingredients, i.e. the anti-TTR antibody and TTR tetramer stabilizer. Associated with such container(s) can be a notice in the form prescribed by a governmental agency regulating the manufacture, use or sale of pharmaceuticals or biological products, which notice reflects approval by the agency of manufacture, use or sale for human administration. The pharmaceutical product of the present invention, preferably in form of a kit is of course particularly suitable for the treatment of a disease or disorder which is accompanied with the presence of mutated, misfolded, misassembled, and/or aggregated TTR, and in particular applicable for the treatment of disorders generally characterized by ATTR which are outlined further below. Alternatively, pharmaceutical product, pharmaceutical pack or kit of the present invention may lack either of the components but comprises the anti-TTR antibody and the TTR tetramer stabilizer only and instead of the other component detailed instructions for use of the pharmaceutical product, pharmaceutical pack or kit in the combination therapy of the invention.

In one embodiment, the combination therapy or pharmaceutical product of the present invention may comprise not only one TTR tetramer stabilizer, but may comprise more than one, for example two TTR tetramer stabilizers. In one embodiment, the pharmaceutical product comprises tafamidis and AG10.

The pharmaceutical product of the present invention can be used in a method of treating ATTR in a subject, i.e. a combination therapy. Thus, the present invention provides a combination therapy comprising administering to a subject in need thereof a therapeutically effective amount of the pharmaceutical product of the present invention.

In one embodiment, the presence and level of amyloidogenic TTR is determined prior to the administration of the anti-TTR antibody and the TTR tetramer stabilizer and the pharmaceutical product of the present invention, respectively. In a preferred embodiment, any suitable anti-TTR antibody that preferentially binds aggregated TTR over physiological TTR can be used. For example, the antibodies disclosed in WO 2015/092077 A1 can be used for diagnosis, preferably antibodies NI-301.37F1, NI-301.28B3 or NI-301.12D3, most preferably NI-301.37F1. Furthermore, antibodies mentioned above, i.e. 14G8, 18C5, 9D5, 5A1, 6C1, XY, 371M, or 313M can be used. Determination of the amyloidogenic TTR amount is preferably performed in a sample (body fluid) from the subject to be treated.

As mentioned above, in one embodiment the pharmaceutical product is present in a single pharmaceutical composition. Thus, in one embodiment of the present invention both components, i.e. the anti-TTR antibody and the TTR tetramer stabilizer are administered simultaneously. This is of particular relevance for the combination of the anti-TTR antibody with AG10 since both components are preferably administered as a single dose.

Preferably, the pharmaceutical product is present as a kit of parts in separate containers. Thus, in one embodiment combination therapy of the present invention, the method of treating ATTR comprises either administering to the subject a therapeutically effective amount of the anti-TTR antibody, and concurrently or subsequently administering to the subject a therapeutically effect amount of the TTR tetramer stabilizer, or administering to the subject a therapeutically effect amount of the TTR tetramer stabilizer, and concurrently or subsequently administering to the subject a therapeutically effect amount of the anti-TTR antibody. Preferably, concurrent or subsequent treatment is performed as long as the first drug is still present in the human body, e.g. plasma at a significant concentration, for example considering the half-life of the drug.

As shown in Example 2, the anti-TTR antibody as defined above induces clearance of TTR fibrils in vivo. Thus, a patient having ATTR and who already receives treatment with one or more TTR tetramer stabilizers in order prevent that more aggregation-prone TTR monomers are generated, may be further treated with the anti-TTR antibody. This has the beneficial effect that in addition to the prevention of TTR monomer generation, the already existing TTR fibrils will be cleared. As mentioned above, TTR tetramer stabilisers are most efficient in the treatment of early stage diseases since these pharmaceuticals do not remove already existing TTR fibrils, but only prevent the generation of aggregation-prone TTR monomers. For example, tafamidis is approved for the treatment of stage 1 ATTR-polyneuropathy by the EMA. Thus, when patients who are already treated with a TTR tetramer stabilizer are further treated with an anti-TTR antibody, it is prudent to expect that this is even efficient in treating patients having ATTR at a later stage. Vice versa, a patient having ATTR and who already receives treatment with an anti-TTR antibody which induces the clearance of TTR fibrils, may be further treated with the TTR tetramer stabilizer as characterized above in order to prevent that new TTR monomers, which tend to aggregate, are generated.

Accordingly, in one embodiment of the present invention, the anti-TTR antibody is used in a method of treating ATTR in a subject who receives a treatment with the TTR tetramer stabilizer. In another embodiment, the TTR tetramer stabilizer is used in a method of treating ATTR in a subject who receives a treatment with the anti-TTR antibody. Put in other words, the present invention relates to a method of treating ATTR by inducing or promoting ATTR fibril clearance through antibody-dependent cellular phagocytosis (ADCP) comprising administering to a subject in need thereof, i.e. patient an anti-TTR antibody and a TTR tetramer stabilizer. In one embodiment the anti-TTR antibody is an antibody as defined hereinbefore and/or the TTR tetramer stabilizer is a stabilizer as defined hereinbefore. The antibody and the stabilizer can be administered concomitantly, sequentially or subsequently.

In this context, the term “subject who receives a treatment” with the TTR stabilizer or anti-TTR antibody includes concurrent treatment as well as sequential treatment as long as the first drug is still present in the human body, e.g. plasma at a significant concentration, for example considering the half-life of the drug. For example, since the mean half-life of tafamidis is approximately 49 hours, administration of the anti-TTR antibody 48 hours after the last dose of tafamidis would be considered as an embodiment of the present invention. On the other hand, if the subject is treated with the second drug, e.g. anti-TTR antibody after the first drug, e.g. tafamidis is degraded or excreted and there is no longer an effective or detectable amount of the first drug in the body, such treatment may not be an embodiment of the present invention.

In a preferred embodiment, the anti-TTR antibody is administered by intravenous infusion and the TTR tetramer stabilizer is administered orally.

As already explained above, the combination therapy is suitable for the treatment of wtATTR and vATTR since both TTR tetramer stabilizer and anti-TTR antibody bind to wtTTR and vTTR and the corresponding aggregated forms of TTR, respectively. In this context, it is to be mentioned that vATTR was formerly known as hereditary/mutant ATTR. ATTR is an autosomal-dominant disorder and is associated with various diseases or conditions which are selected from the group consisting of ATTR-Cardiomyopathy, Familial Amyloid Polyneuropathy (FAP) (also known as TTR-polyneuropathy), Senile Systemic Amyloidosis (SSA), systemic familial amyloidosis, leptomeningeal/Central Nervous System (CNS) amyloidosis including Alzheimer's disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia.

Thus, in one embodiment of the present invention, the pharmaceutical product and the combination therapy, respectively of the present invention as well as the anti-TTR antibody and the TTR tetramer stabilizer when administered to a patient who already receives treatment are used in the treatment of any one of the diseases mentioned above.

In a preferred embodiment, the disease is ATTR-cardiomyopathy. ATTR-cardiomyopathy is an age-associated disease of the heart characterized by intramyocardial deposition of misfolded and aggregated TTR. The causative amyloid deposits in the heart may be composed of either wtTTR or vTTR, the latter associated with numerous mutations in the TTR gene. The accumulation of amyloid results in increased ventricular wall thickness and severely impacts heart function and survival. The disease manifests predominantly in men ≥60 years of age and can be diagnosed using echocardiography, scintigraphy and biopsies. The prevalence of the disease is currently unknown despite the fact that diagnosis tools are widely accessible. It is estimated that today only 1% or less of the patients are being diagnosed.

There are different disease stages of ATTR, i.e. early, advance or late-onset disease. It is prudent to expect that the pharmaceutical product of the present invention, i.e. the combination therapy is useful for treating patients having ATTR in an early disease state, as well as patients having ATTR in an advanced or late stage. In particular, as mentioned above, the anti-TTR antibody efficiently removes TTR fibrils from diseased tissue and thus, even at a late disease stage, it can be expected that the diseased tissue and the respective organ, which is in case of ATTR-cardiomyopathy the heart, recovers after treatment with the anti-TTR antibody. At the same time, the TTR tetramer stabilizer prevents the formation of new aggregated TTR species so that a beneficial effect on the patient's health is highly likely.

Although of course any anti-TTR antibody against misfolded TTR which is capable of facilitating removal of amyloidogenic TTR and any TTR tetramer stabilizer can be combined to the pharmaceutical product of the present invention, the following combinations are preferred:
Antibody NI-301.37F1 with tafamidis
Antibody NI-301.28B3 with tafamidis
Antibody NI-301.12D3 with tafamidis
Antibody 14G8 with tafamidis
Antibody 18C5 with tafamidis
Antibody 9D5 with tafamidis
Antibody 5A1 with tafamidis
Antibody 6C1 with tafamidis
Antibody XY with tafamidis
Antibody 371M with tafamidis
Antibody 313M with tafamidis
Antibody NI-301.37F1 with AG10
Antibody NI-301.28B3 with AG10
Antibody NI-301.12D3 with AG10
Antibody 14G8 with AG10
Antibody 18C5 with AG10
Antibody 9D5 with AG10
Antibody 5A1 with AG10
Antibody 6C1 with AG10
Antibody XY with AG10
Antibody 371M with AG10
Antibody 313M with AG10
Antibody NI-301.37F1 with diflunisal
Antibody NI-301.37F1 with diflunisal
Antibody NI-301.28B3 with diflunisal
Antibody NI-301.12D3 with diflunisal
Antibody 14G8 with diflunisal
Antibody 18C5 with diflunisal
Antibody 9D5 with diflunisal
Antibody 5A1 with diflunisal
Antibody 6C1 with diflunisal
Antibody XY with diflunisal
Antibody 371M with diflunisal
Antibody 313M with diflunisal
Antibody NI-301.37F1 with tafamidis and AG10
Antibody NI-301.28B3 with tafamidis and AG10
Antibody NI-301.12D3 with tafamidis and AG10
Antibody 14G8 with tafamidis and AG10
Antibody 18C5 with tafamidis and AG10
Antibody 9D5 with tafamidis and AG10
Antibody 5A1 with tafamidis and AG10
Antibody 6C1 with tafamidis and AG10
Antibody XY with tafamidis and AG10
Antibody 371M with tafamidis and AG10
Antibody 313M with tafamidis and AG10

A particular preferred embodiment of the present invention is the combination of antibody NI-301.37F1 with tafamidis.

Although of course each combination can be used for the treatment of ATTR in general, each combination can be used for the treatment of ATTR-cardiomyopathy and/or ATTR-polyneuropathy:
Antibody NI-301.37F1 with tafamidis for use in the treatment of ATTR-cardiomyopathy
Antibody NI-301.37F1 with tafamidis for use in the treatment of ATTR-polyneuropathy
Antibody NI-301.28B3 with tafamidis for use in the treatment of ATTR-cardiomyopathy
Antibody NI-301.28B3 with tafamidis for use in the treatment of ATTR-polyneuropathy
Antibody NI-301.12D3 with tafamidis for use in the treatment of ATTR-cardiomyopathy
Antibody NI-301.12D3 with tafamidis for use in the treatment of ATTR-polyneuropathy
Antibody 14G8 with tafamidis for use in the treatment of ATTR-cardiomyopathy
Antibody 14G8 with tafamidis for use in the treatment of ATTR-polyneuropathy
Antibody 18C5 with tafamidis for use in the treatment of ATTR-cardiomyopathy
Antibody 18C5 with tafamidis for use in the treatment of ATTR-polyneuropathy
Antibody 9D5 with tafamidis for use in the treatment of ATTR-cardiomyopathy
Antibody 9D5 with tafamidis for use in the treatment of ATTR-polyneuropathy
Antibody 5A1 with tafamidis for use in the treatment of ATTR-cardiomyopathy
Antibody 5A1 with tafamidis for use in the treatment of ATTR-polyneuropathy
Antibody 6C1 with tafamidis for use in the treatment of ATTR-cardiomyopathy
Antibody 6C1 with tafamidis for use in the treatment of ATTR-polyneuropathy
Antibody XY with tafamidis for use in the treatment of ATTR-cardiomyopathy
Antibody XY with tafamidis for use in the treatment of ATTR-polyneuropathy
Antibody 371M with tafamidis for use in the treatment of ATTR-cardiomyopathy
Antibody 371M with tafamidis for use in the treatment of ATTR-polyneuropathy
Antibody 313M with tafamidis for use in the treatment of ATTR-cardiomyopathy
Antibody 313M with tafamidis for use in the treatment of ATTR-polyneuropathy
Antibody NI-301.37F1 with AG10 for use in the treatment of ATTR-cardiomyopathy
Antibody NI-301.37F1 with AG10 for use in the treatment of ATTR-polyneuropathy
Antibody NI-301.28B3 with AG10 for use in the treatment of ATTR-cardiomyopathy
Antibody NI-301.28B3 with AG10 for use in the treatment of ATTR-polyneuropathy
Antibody NI-301.12D3 with AG10 for use in the treatment of ATTR-cardiomyopathy
Antibody NI-301.12D3 with AG10 for use in the treatment of ATTR-polyneuropathy
Antibody 14G8 with AG10 for use in the treatment of ATTR-cardiomyopathy
Antibody 14G8 with AG10 for use in the treatment of ATTR-polyneuropathy
Antibody 18C5 with AG10 for use in the treatment of ATTR-cardiomyopathy
Antibody 18C5 with AG10 for use in the treatment of ATTR-polyneuropathy
Antibody 9D5 with AG10 for use in the treatment of ATTR-cardiomyopathy
Antibody 9D5 with AG10 for use in the treatment of ATTR-polyneuropathy
Antibody 5A1 with AG10 for use in the treatment of ATTR-cardiomyopathy
Antibody 5A1 with AG10 for use in the treatment of ATTR-polyneuropathy
Antibody 6C1 with AG10 for use in the treatment of ATTR-cardiomyopathy
Antibody 6C1 with AG10 for use in the treatment of ATTR-polyneuropathy
Antibody XY with AG10 for use in the treatment of ATTR-cardiomyopathy
Antibody XY with AG10 for use in the treatment of ATTR-polyneuropathy
Antibody 371M with AG10 for use in the treatment of ATTR-cardiomyopathy
Antibody 371M with AG10 for use in the treatment of ATTR-polyneuropathy
Antibody 313M with AG10 for use in the treatment of ATTR-cardiomyopathy
Antibody 313M with AG10 for use in the treatment of ATTR-polyneuropathy
Antibody NI-301.37F1 with diflunisal for use in the treatment of ATTR-cardiomyopathy
Antibody NI-301.37F1 with diflunisal for use in the treatment of ATTR-polyneuropathy
Antibody NI-301.28B3 with diflunisal for use in the treatment of ATTR-cardiomyopathy
Antibody NI-301.28B3 with diflunisal for use in the treatment of ATTR-polyneuropathy
Antibody NI-301.12D3 with diflunisal for use in the treatment of ATTR-cardiomyopathy
Antibody NI-301.12D3 with diflunisal for use in the treatment of ATTR-polyneuropathy
Antibody 14G8 with diflunisal for use in the treatment of ATTR-cardiomyopathy
Antibody 14G8 with diflunisal for use in the treatment of ATTR-polyneuropathy
Antibody 18C5 with diflunisal for use in the treatment of ATTR-cardiomyopathy
Antibody 18C5 with diflunisal for use in the treatment of ATTR-polyneuropathy
Antibody 9D5 with diflunisal for use in the treatment of ATTR-cardiomyopathy
Antibody 9D5 with diflunisal for use in the treatment of ATTR-polyneuropathy
Antibody 5A1 with diflunisal for use in the treatment of ATTR-cardiomyopathy
Antibody 5A1 with diflunisal for use in the treatment of ATTR-polyneuropathy
Antibody 6C1 with diflunisal for use in the treatment of ATTR-cardiomyopathy
Antibody 6C1 with diflunisal for use in the treatment of ATTR-polyneuropathy
Antibody XY with diflunisal for use in the treatment of ATTR-cardiomyopathy
Antibody XY with diflunisal for use in the treatment of ATTR-polyneuropathy
Antibody 371M with diflunisal for use in the treatment of ATTR-cardiomyopathy
Antibody 371M with diflunisal for use in the treatment of ATTR-polyneuropathy
Antibody 313M with diflunisal for use in the treatment of ATTR-cardiomyopathy
Antibody 313M with diflunisal for use in the treatment of ATTR-polyneuropathy
Antibody NI-301.37F1 with tafamidis and AG10 for use in the treatment of ATTR-cardiomyopathy
Antibody NI-301.37F1 with tafamidis and AG10 for use in the treatment of ATTR-polyneuropathy
Antibody NI-301.28B3 with tafamidis and AG10 for use in the treatment of ATTR-cardiomyopathy
Antibody NI-301.28B3 with tafamidis and AG10 for use in the treatment of ATTR-polyneuropathy
Antibody NI-301.12D3 with tafamidis and AG10 for use in the treatment of ATTR-cardiomyopathy
Antibody NI-301.12D3 with tafamidis and AG10 for use in the treatment of ATTR-polyneuropathy
Antibody 14G8 with tafamidis and AG10 for use in the treatment of ATTR-cardiomyopathy
Antibody 14G8 with tafamidis and AG10 for use in the treatment of ATTR-polyneuropathy
Antibody 18C5 with tafamidis and AG10 for use in the treatment of ATTR-cardiomyopathy
Antibody 18C5 with tafamidis and AG10 for use in the treatment of ATTR-polyneuropathy
Antibody 9D5 with tafamidis and AG10 for use in the treatment of ATTR-cardiomyopathy
Antibody 9D5 with tafamidis and AG10 for use in the treatment of ATTR-polyneuropathy
Antibody 5A1 with tafamidis and AG10 for use in the treatment of ATTR-cardiomyopathy
Antibody 5A1 with tafamidis and AG10 for use in the treatment of ATTR-polyneuropathy
Antibody 6C1 with tafamidis and AG10 for use in the treatment of ATTR-cardiomyopathy
Antibody 6C1 with tafamidis and AG10 for use in the treatment of ATTR-polyneuropathy
Antibody XY with tafamidis and AG10 for use in the treatment of ATTR-cardiomyopathy
Antibody XY with tafamidis and AG10 for use in the treatment of ATTR-polyneuropathy
Antibody 371M with tafamidis and AG10 for use in the treatment of ATTR-cardiomyopathy
Antibody 371M with tafamidis and AG10 for use in the treatment of ATTR-polyneuropathy
Antibody 313M with tafamidis and AG10 for use in the treatment of ATTR-cardiomyopathy
Antibody 313M with tafamidis and AG10 or use in the treatment of ATTR-polyneuropathy

A particular preferred embodiment of the present invention is the combination of antibody NI-301.37F1 with tafamidis for use in the treatment of ATTR-cardiomyopathy.

Of course, when referring to the above-mentioned antibodies, their fully human or humanized version, respectively, is meant encompassing corresponding human, humanized and human-like antibodies that compete for binding to human misfolded/aggregated TTR with said reference antibody.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific example which are provided herein for purposes of illustration only and is not intended to limit the scope of the invention.

### EXAMPLES

### Materials and Methods

### TTR Protein Aggregates

Human wild-type TTR protein obtained by purification from human plasma, and wild-type and mutant TTR proteins obtained by recombinant expression were used in both native and misfolded-aggregated conformations for the identification of TTR-targeting antibodies. The misfolded-aggregated conformations were produced in vitro under acidic conditions, using a procedure similar to the one described in Colon W. et al, Biochemistry, 31 (1992), 8654-8660, with minor modifications. Plasma-purified wtTTR was provided as a solution at a concentration of 2 mg/mL in PBS buffer.

### Anti-TTR Antibody

The mouse chimeric antibody ch.NI-301.37F1 was generated based on the human-derived monoclonal antibody NI-301.37F1 disclosed in the international application WO 2015/092077 A1. The mouse chimeric variant was designed to contain the human variable domains of NI-301.37F1 in murine constant domain backbones. In particular, the amino acid sequences of the variable heavy (VH) and light (VL) chain of human-derived monoclonal antibody NI-301.37F1 are disclosed in WO 2015/092077 A1 in FIG. 1 with SEQ ID NO: 10 and 53, respectively, corresponding to SEQ ID NO: 2 and 6 of the present sequence listing for the VH chain and SEQ ID NO: 12 corresponding to SEQ ID NO: 4 of the present sequence listing for the VL chain, while the mouse heavy chain constant domain corresponds to Uniprot entry P01863 and the mouse light chain constant domain corresponds to Uniprot entry P01837. In brief, gene synthesis was used to produce a synthetic heavy chain gene comprising the sequence coding for the human variable heavy chain of NI-301.37F1 followed by the sequence coding for a murine IgG2a constant heavy chain (cf. sequence mur.37F1 H), and a synthetic light chain gene comprising the sequence coding for the human variable chain of NI-301.37F1 followed by the sequence coding for a murine constant kappa light chain (cf. mur.37F1 L). These 2 genes were then sub-cloned into suitable expression vectors that were used for the transfection of CHO cells. Ch.NI-301.37F1 antibody was purified from the cell culture medium using standard processes as described in WO 2015/092077 A1 including purification of the antibody by chromatography on protein A column.

### TTR Tetramer Stabilizers

Tafamidis (Mw: 308.1 g/mol) was prepared at 100 μg/mL (325 μM) in 10% EtOH, 10% PS80 and 80% PBS pH7.4.

Diflunisal (Mw: 250.2 g/mol) was prepared at 1.875 mg/mL in 12.5% EtOH, 12.5% PS80 and 75% PBS.

AG10 (Mw: 292.3 g/mol) was prepared at 200 μg/mL in 10% EtOH, 10% PS80 and 80% PBS pH7.4 as follow. 2 mg of AG10 was solubilized in 1 mL pure ethanol, supplemented with 1 mL polysorbate 80 and diluted progressively with 8 mL PBS under constant stirring.

### TTR Stabilizer Solutions:

| | | Identification |
|---|---|---|
| Material | Source | number |
| Tafamidis | Biosynth-Carbosynth | FD27988 |
| Diflunisal | Sigma | D3281 |
| AG10 | Aquila Pharmatech | AN16167 |
| Ethanol 100% | Sigma | 02870 |
| Polysorbate 80 | Sigma | P5188 |
| PBS pH 7.4 1× | Gibco | 10010-015 |

### ATTR Animal Model

Patient-derived amyloid xenograft (PDAX) mice have been prepared and used as disclosed in WO 2020/094883 A1.

### ELISA

96 well microplates were coated for 1 hour at 37° C. with mis.WT-TTR aggregates diluted to a concentration of 10 μg/mL in PBS buffer pH7.4. Non-specific binding sites were blocked for 1 hour at room temperature (RT) with a blocking buffer containing 2% bovine serum albumin (BSA) and 0.1% tween-20 in PBS buffer. Human NI-301.37F1 antibody was diluted in duplicates to the indicated concentrations in PBS supplemented with tafamidis or diflunisal at 10 μg/mL or corresponding concentration of vehicle buffer, and incubated overnight at 4° C. Binding was determined using an anti-human IgG antibody conjugated with horseradish peroxidase (HRP), followed by measurement of HRP activity in a standard colorimetric assay.

### EC50 Determination

Data were analyzed with the Prism software from GraphPad. EC50 values were estimated using non-linear regression of individual data points using log(agonist) versus response model with variable slope. Data fitting was performed with the least square regression method.

### Reagents and Instrumentation for EC50 ELISA:

| | | Identification |
|---|---|---|
| Description | Source | number |
| ½ Area ELISA Plates | Corning | 3690 |
| Phosphate buffered saline pH | Gibco | 10010-015 |
| 7.4 (PBS) | | |
| Tween-20 | Sigma | P1379-500ML |
| Bovine Serum Albumin (BSA) | Sigma | A8022 |
| Anti-human IgG HRP-labeled | Jackson | 709-036-098 |
| antibody | Immunoresearch | |
| TMB | Pierce | 34021 lot UF284180 |
| Stop solution | Invitrogen | SS04 lot 206369000 |
| VarioScan Lux | ThermoFischer | 2016-03-00700 |
| | Scientific | |
| Prism | GraphPad | v.8 |

### Binding Kinetic Assay

The antibody NI-301.37F1 was loaded on anti-human capture sensors at 5 μg/mL in PBS for 5 min at 25° C. The binding assay was performed with soluble WT-TTR amyloid aggregates and tetrameric WT-TTR. A baseline was performed in 1× kinetic buffer for 3 min, the association was measured for 10 min and the dissociation for another 10 min in 1× kinetic buffer, all steps performed at 25° C. with shaking at 1000 rpm. A reference sample was also included consisting in 1× kinetic buffer.

Data were analyzed in ForteBio Data Analysis 8.2 software, using reference subtraction and interstep correction between association and dissociation.

### Reagents and Instrumentation for Binding Kinetic Assay:

| | | Identification |
|---|---|---|
| Description | Source | number |
| Anti-human capture (AHC) sensors | Molecular Devices | 18-5060 |
| Octet Red96 | Molecular Devices | OCTETRED96 |
| Kinetic buffer 10× | Molecular Devices | 18-1092 |

### Example 1: Preserved NI-301.37F1 Target Binding in Presence of TTR Tetramer Stabilizers

For the assessment of the binding affinity of anti-TTR antibody in the presence of TTR tetramer stabilizer direct ELISA and EC₅₀ determination has been substantially performed in Example 3 of WO 2015/092077 A1. ELISA plates were coated for 1 h at 37° C. with amyloid wtTTR aggregates and blocked for 1 h at room temperature with blocking buffer (PBS pH 7.4, 1% BSA and 0.1% polysorbate 20).

Tafamidis and diflunisal were dissolved in vehicle (80% PBS pH 7.4, 10% ethanol, 10% polysorbate 80) and antibody NI-301.37F1 dilution series were prepared with tafamidis or diflunisal at a final concentration of 10 μg/mL or a corresponding volume of the vehicle. The concentration of 10 μg/mL is 40% above the maximal peak concentration in patients treated with tafamidis at 80 mg per day (˜6 μg/mL). NI-301.37F1 dilution series were incubated in the ELISA plates overnight at 4° C., and, after washes, NI-301.37F1 was detected using an horseradish peroxidase (HRP)-coupled anti-human IgG antibody and standard detection methods. Data were analyzed with the Prism software from GraphPad. EC₅₀ values were estimated using non-linear regression of individual data points using log(agonist) versus response model with variable slope. Data fitting was performed with the least square regression method.

The results showed that antibody NI-301.37F1 binding to amyloidogenic TTR is maintained in the presence of TTR stabilizers such as tafamidis and diflunisal.

Patient-derived amyloid xenograft (PDAX) mouse model has been prepared as disclosed in WO 2020/094883 A1.

### Example 2: NI-301.37F1-TTR Stabilizer Drug Combination is Active In Vivo

Amyloidogenic TTR-grafted mice (PDAX mouse model) received immediately a single administration of the mouse chimeric NI-301.37F1 variant (ch.NI-301.37F1) at 10 mg/kg i.v. (intravenous injection) or a corresponding isotype antibody as negative control. Tafamidis and diflunisal were dissolved in the vehicle (80% PBS pH 7.4, 10% ethanol, 10% polysorbate 80). Tafamidis was administered at 1 mg/kg daily i.p. (intraperitoneal injection). This dose level corresponds to 33% of the rat equivalent of the maximum recommended human dose (MRHD). Diflunisal, which has a much faster elimination and lower affinity for TTR, was administered twice daily at 19 mg/kg i.p in the morning and evening.

Animals were sacrificed 96 hours after fibril grafting and antibody administration to collect skin tissues and the attached amyloidogenic TTR fibril grafts. The grafts were sectioned and six non-consecutive sections were used to quantify the remaining amount of amyloidogenic TTR fibrils in the graft by immunohistochemistry (IHC). TTR IHC was performed using the commercial TTR antibody Dako A0002 for staining. In particular, immunostaining was performed according to standard procedures and as disclosed in WO 2020/094883 A1. Amyloidogenic TTR amount within the graft were quantified using an automated image analysis process. Data were analyzed with a one-way ANOVA and Sidak's test for group by group comparisons by reference to the isotype+vehicle group.

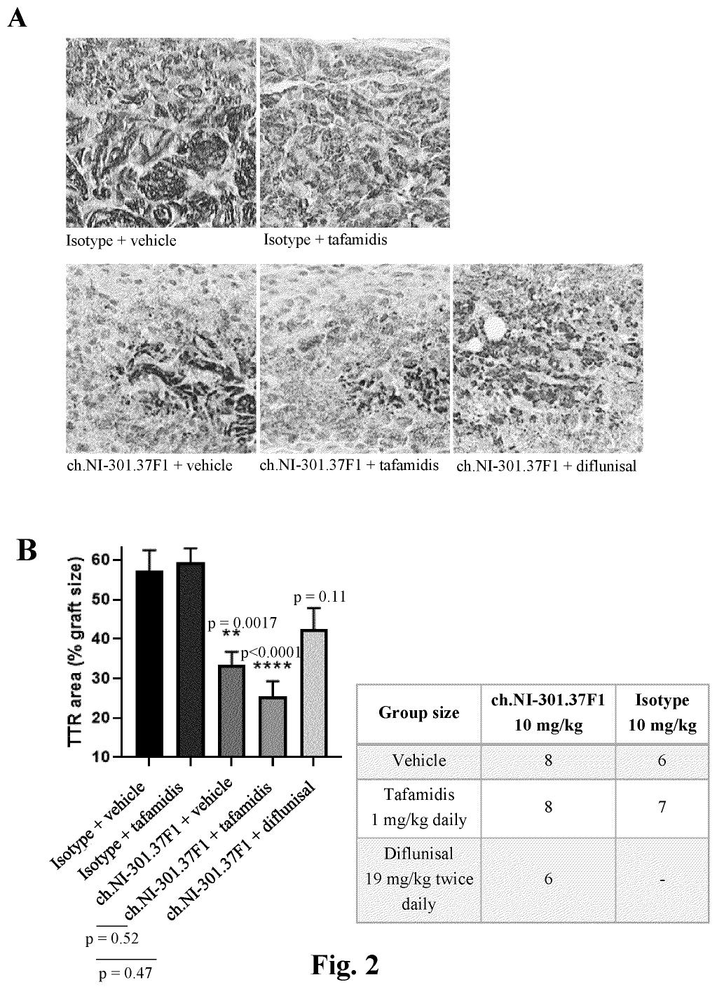

The results showed that ch.NI-301.37F1 fibril removal activity was maintained upon co-treatment with tafamidis and diflunisal in vivo, with a pronounced effect in the presence of tafamidis which seems to even enhance the in vivo clearance of pathological TTR fibrils induced by the antibody, while co-treatment with diflunisal resulted in a somewhat lower but still significant clearance rate; see FIG. 2 and Example 3.

### Example 3: Antibody-Mediated Fibril Removal Activity In Vivo Requires an Active Fc Domain

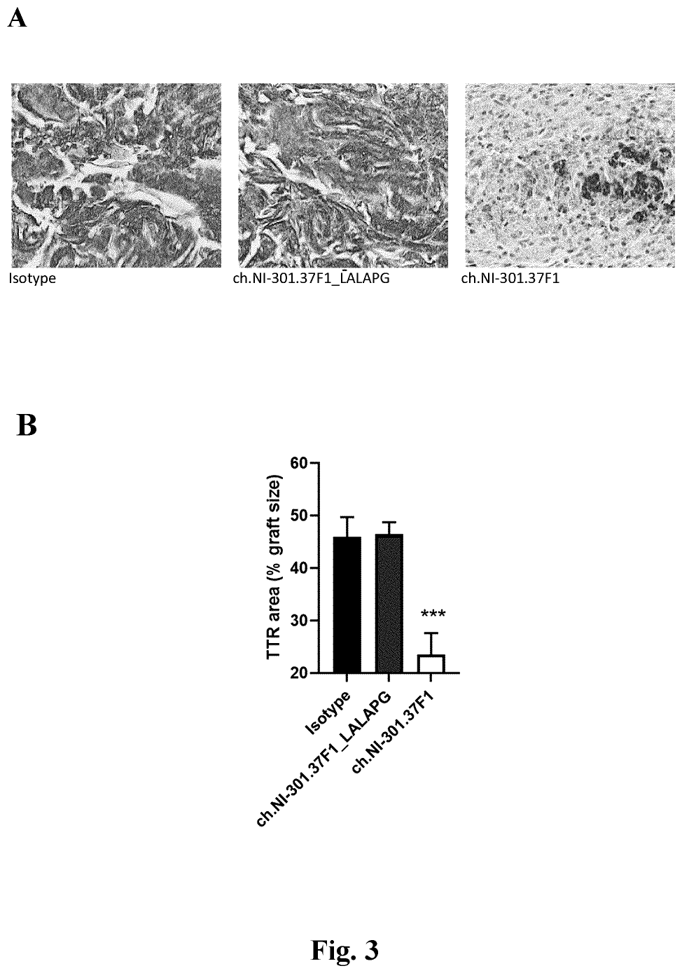

Amyloidogenic TTR-grafted mice (PDAX mouse model) were treated with ch37F1 antibody at 0.15, 0.5, 1.5 and 5 mg/kg i.v. or with the Fc-inactive variant ch37F1-LALAPG or the isotype control antibody at 5 mg/kg. The combination of the L234A, L235A and P329G amino-acid substitutions are sufficient to virtually abolish binding to FcgR1, 2, 3 and 4 and complement C1q binding while preserving antibody stability (Lo et al., J. Biol. Chem. 292 (2017), 3900-3908). After 5 days, ATTR fibril grafts were collected and the remaining amount of ATTR fibrils quantified using IHC as performed in Example 2. As shown in FIG. 3, 5 days after fibril graft and treatment administration, ATTR fibrils covered 46% of the graft area in isotype treated mice, 23.6% in ch.NI-301.37F1 treated mice, and 46.5% in mice treated with ch.NI-301.37F1-LALAPG. The fibril removal activity observed with ch.NI-301.37F1 compared to isotype was statistically significant (p<0.001, 1-way ANOVA and Dunnetts' multiple comparisons test). In contrast, no fibril removal activity was observed in mice treated with ch.NI-301.37F1-LALAPG compared to isotype treated mice. These results indicate that an active Fc domain is required for antibody-mediated fibril removal by phagocytic immune cells in vivo.

### Example 4: Binding Kinetics of Anti-TTR Antibody in Presence of a TTR Stabilizing Agent

### 4.1 NI-301.37F1 Binding Selectivity in Presence of Tafamidis and Diflunisal Using Biolayer Interferometry (BLI)

NI-301.37F1 absent binding to TTR tetramers was investigated in presence and absence of TTR stabilizers using BLI. Soluble ATTR oligomers were included in this experiment to serve as positive controls.

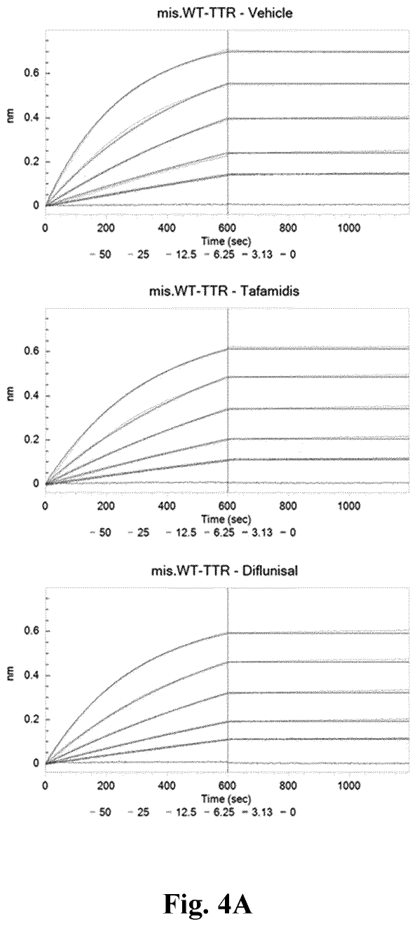

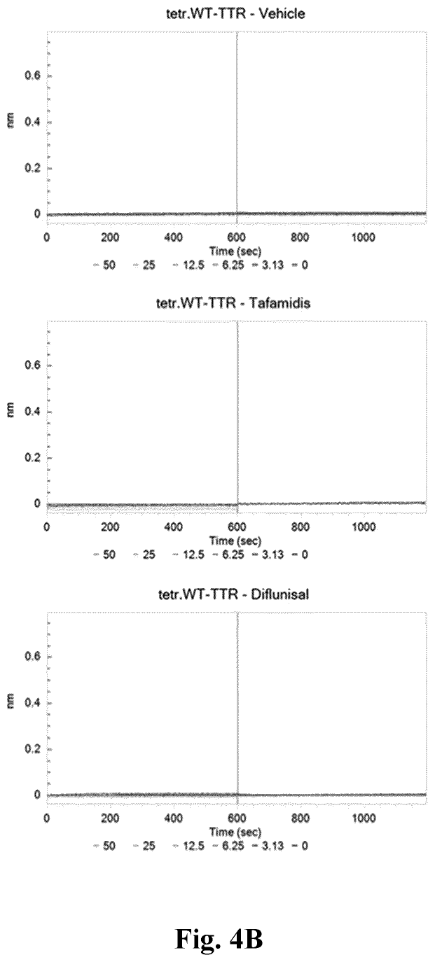

The results indicated that NI-301.37F1 did not bind TTR tetramers in solution, which is consistent with prior results. In addition, the results indicated that NI-301.37F1 did not bind TTR-tafamidis and TTR-diflunisal complexes (FIG. 4A, 4B).

### 4.2 NI-301.37F1 Binding Kinetics and Selectivity in Presence of AG10 and Diflunisal Using BLI

NI-301.37F1 binding to ATTR oligomers and TTR tetramers was evaluated in presence of the TTR stabilizers AG10 and diflunisal at 5 μg/mL, or in presence of corresponding vehicle buffer using BLI.

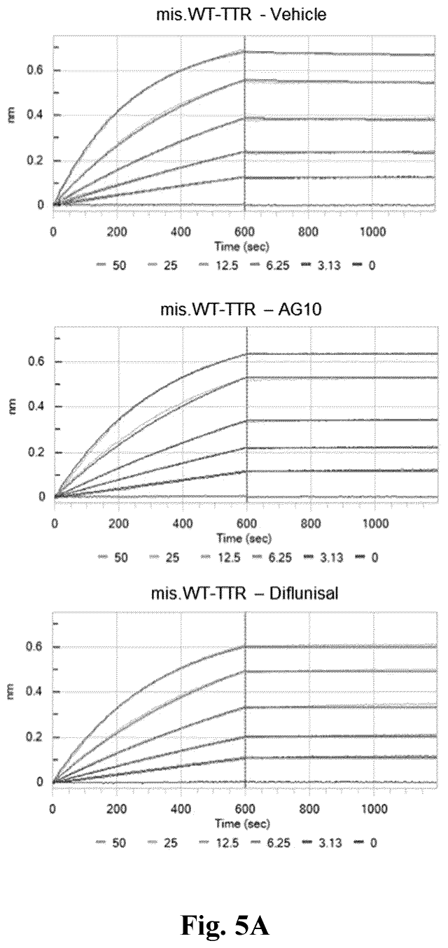

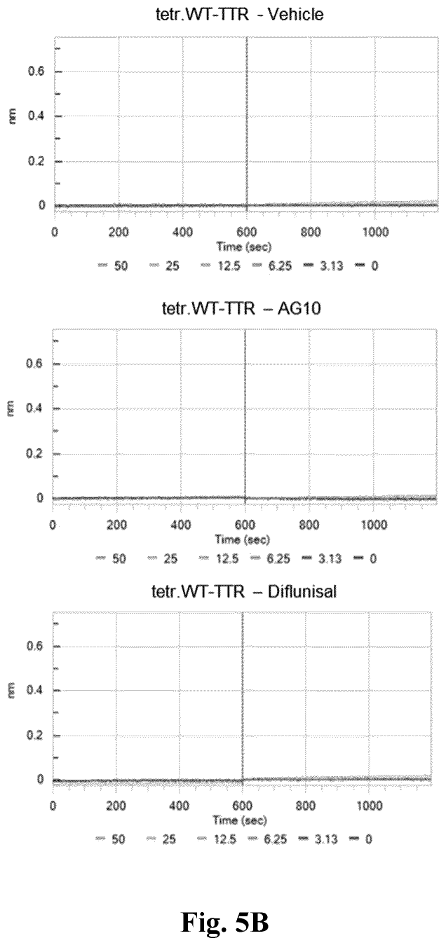

The results indicated that NI-301.37F1 binding to ATTR oligomers in solution was virtually identical in presence or absence of AG10 or diflunisal (FIG. 5A, 5B).

### CONCLUSION

NI-301.37F1 binds ATTR amyloid with similar affinity in presence or absence of the TTR stabilizers tafamidis, diflunisal and AG10.

Likewise, NI-301.37F1 binding selectivity, characterized by its absent binding to TTR tetramers, is maintained in presence of TTR stabilizers.

1. A combination therapy for use in a method of treating transthyretin amyloidosis (ATTR) comprising an anti-transthyretin (TTR) antibody and a TTR tetramer stabilizer, wherein the antibody has an active Fc domain and is capable of inducing ATTR fibril clearance through antibody-dependent cellular phagocytosis (ADCP).

2. The combination therapy for use according to claim 1, wherein the antibody is of the IgG1 class or isotype.

3. The combination therapy for use according to any one of claim 1 or 2, wherein the antibody is a human-derived antibody.

4. The combination therapy for use according to any one of claims 1 to 3, wherein the antibody is capable of binding a TTR epitope which comprises or consists of the amino acid sequence WEPFA (SEQ ID NO: 7) and wherein the antibody comprises in its variable region or binding domain:
(i) the six CDRs of the VH and VL variable region selected from:
VH-CDR1: positions 31-35 of SEQ ID NO: 2
VH-CDR2: positions 52-67 of SEQ ID NO: 2
VH-CDR3: positions 100-109 of SEQ ID NO: 2
VL-CDR1: positions 24-34 of SEQ ID NO: 4
VL-CDR2: positions 50-56 of SEQ ID NO: 4
VL-CDR3: positions 89-97 of SEQ ID NO: 4, or wherein one or more of the CDRs may comprise one or two amino acid substitutions;
VH-CDR1: positions 31-35 of SEQ ID NO: 6
VH-CDR2: positions 52-67 of SEQ ID NO: 6
VH-CDR3: positions 100-109 of SEQ ID NO: 6
VL-CDR1: positions 24-34 of SEQ ID NO: 4
VL-CDR2: positions 50-56 of SEQ ID NO: 4
VL-CDR3: positions 89-97 of SEQ ID NO: 4, or wherein one or more of the CDRs may comprise one or two amino acid substitutions; or
(ii) a VH chain comprising an amino acid sequence at least 90% identical to SEQ ID NO: 2 or SEQ ID NO: 6 and a VL chain region comprising an amino acid sequence at least 90% identical to SEQ ID NO: 4.

5. The combination therapy for use according to any one of claims 1 to 4, wherein the antibody comprises in its variable region or binding domain the amino acid sequences of VH and VL chain of SEQ ID NO: 2 and SEQ ID NO: 4 or SEQ ID NO: 6 and SEQ ID NO: 4.

6. The combination therapy for use according to any one of claims 1 to 5, wherein the stabilizer is tafamidis, diflunisal, AG10 or a combination thereof.

7. The combination therapy for use according to any one of claims 1 to 5, wherein the stabilizer is tafamidis.

8. The combination therapy for use according to any one of claims 1 to 7, wherein the anti-TTR antibody is designed to be administered at a dose of 1, 3, 10, 30 or 60 mg/kg or a dose in between every 2-4 weeks and the TTR tetramer stabilizer in a clinically active concentration.

9. The combination therapy for use according to claim 8, wherein the stabilizer is tafamidis or tafamidis meglumine and designed to be administered at a dose of 1, 5, 15, or 30 mg/kg/day or a dose in between, optionally at a dose of 20 or 80 mg/day tafamidis meglumine and 12.2 or 61 mg/day tafamidis, respectively.

10. The combination therapy for use according to claim 8, wherein the stabilizer is AG10 and designed to administered at a dose of 50, 150, 300, 400 or 800 mg or a dose in between, optionally twice daily.

11. The combination therapy for use according to claim 8, wherein the stabilizer is diflunisal and is designed to be administered at a dose of 250 mg, optionally twice daily.

12. The combination therapy for use according to any one of claims 1 to 11, wherein the anti-TTR antibody is administered concurrently with, prior to, or subsequent to the stabilizer.

13. The combination therapy for use according to any one of claims 1 to 12, wherein the antibody and the stabilizer are present in a pharmaceutical product or as a kit of parts in separate containers.

14. The combination therapy for use according to claim 13, wherein the antibody is present as liquid formulation in an infusion bottle or bag and/or the stabilizer is present in a tablet or capsule for oral administration.

15. A pharmaceutical product or kit of parts as defined in claim 13 or 14.

16. An anti-TTR antibody as defined in any one of the preceding claims for use in treating ATTR in a subject who receives a treatment with the TTR tetramer stabilizer as defined in any one of the preceding claims or a TTR tetramer stabilizer as defined in any one of the preceding claims for use in treating ATTR in a subject who receives a treatment with the anti-TTR antibody as defined in any one of the preceding claims.

17. A method of treating ATTR by inducing or promoting ATTR fibril clearance through antibody-dependent cellular phagocytosis (ADCP) comprising administering to a patient in need thereof an anti-transthyretin (TTR) antibody and a TTR tetramer stabilizer.

18. The method of claim 17, wherein the anti-TTR antibody is an antibody as defined in any one of the preceding claims and/or the TTR tetramer stabilizer is a stabilizer as defined in any one of the preceding claims.

19. The method of claim 17 or 18, wherein the antibody and the stabilizer are administered concomitantly, sequentially or subsequently.

20. The combination therapy for use according to any one of claims 1 to 14, or the anti-TTR antibody, the TTR tetramer stabilizer for use according to claim 16, or the method of any one of claims 17 to 19, wherein ATTR is wild-type (wtATTR) or variant ATTR (vATTR) and wherein the ATTR is associated with a disease or condition selected from the group consisting of ATTR polyneuropathy, ATTR cardiomyopathy, Familial Amyloid Polyneuropathy (FAP), Senile Systemic Amyloidosis (SSA), systemic familial amyloidosis, leptomeningeal/Central Nervous System (CNS) amyloidosis, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia.
